(19) United States
(12) Patent Application Publication
(10) Pub. No.: US 2026/0325144 A1
(43) Pub. Date: Oct. 22, 2026

(54) ORTHOPAEDIC PLANNING SYSTEMS, INSTRUMENTATION AND METHODS OF REPAIR FOR RESTORING BONE LOSS

(71) Applicant: ARTHREX, INC., Naples, FL (US)

(72) Inventors: Kevin Donnelly, Belfast (GB); Brendan Gallagher, Belfast (GB); Alex Campagnoli, Munich (DE); Benedikt Beyer, Germering (DE); Steven Jim DeLeon, San Antonio, TX (US); Nick Metcalfe, Bonita Springs, FL (US)

(21) Appl. No.: 19/529,327

(22) Filed: Feb. 4, 2026

Related U.S. Application Data
(60) Provisional application No. 63/760,162, filed on Feb. 19, 2025.

(51) Int. Cl. A61B 17/16; A61B 17/00; A61B 17/15; A61B 17/17; A61B 17/56; A61B 34/10; A61B 90/00; A61F 2/46

(52) U.S. Cl. CPC A61B 17/1635; A61B 17/15; A61B 17/1778; A61B 17/56; A61B 34/10; A61B 90/03; A61F 2/4644; A61B 2017/00477; A61B 2017/564; A61B 2034/108; A61B 2090/036

(57) ABSTRACT

This disclosure relates to planning systems, methods and instrumentation. The planning systems, methods and instrumentation disclosed herein may be utilized for planning orthopaedic procedures to restore functionality to a joint, may include determining bone loss along or otherwise adjacent to an articular surface of a bone. Instrumentation may be formed to prepare the bone. The articular surface may be repaired, which may include utilizing the instrumentation and planning systems to position and secure a bone graft along a portion of the bone associated with the bone loss.

### CROSS-REFERENCE TO RELATED APPLICATIONS

This application claims the benefit of U.S. Provisional Application No. 63/760,162, filed Feb. 19, 2025, which is incorporated herein by reference in its entirety.

### BACKGROUND

This disclosure relates to orthopaedic procedures and, more particularly, to systems and methods for planning and implementing the repair of bone defects and restoration of functionality to a joint.

Many bones of the human musculoskeletal system include articular surfaces. The articular surfaces articulate relative to other bones to facilitate different types and degrees of joint movement. The articular surfaces can erode or experience bone loss over time due to repeated use or wear or may fracture due to a traumatic impact. These types of bone defects may cause joint instability and pain. Some techniques utilize a bone graft and/or implant to repair a defect adjacent the articular surfaces.

The bone deficiency may occur along an articular surface of a glenoid. The surgeon may utilize a Latarjet procedure to repair the defect. The procedure may include performing an osteotomy to harvest a portion of the coracoid process and then positioning the harvested bone along the defect area.

### SUMMARY

This disclosure relates to planning systems, methods and instrumentation. The planning systems, methods and instrumentation may be utilized for planning and implementing orthopaedic procedures to restore functionality to a joint, including determining bone loss adjacent to an articular surface of a bone, forming instrumentation suitable for preparing the bone, and/or repairing the articular surface which may include securing a bone graft along a position of the bone associated with the bone loss.

A guide for an orthopaedic procedure according to an implementation may include a first reference member and/or a second reference member. The first reference member may include a first body having a first contact surface dimensioned according to a first patient-specific contour, which may be associated with a first non-articular surface of an anatomy. The first body may include at least one guide feature dimensioned to guide a surgical instrument. The at least one guide feature may include a resection surface that may extend along a resection plane. The second reference member may be spaced apart from the first reference member. The second reference member may include a second body having a second contact surface dimensioned according to a second patient-specific contour, which may be associated with an articular surface of the anatomy. A linking member may interconnect the first and second reference members.

A guide for an orthopaedic procedure according to an implementation may include a first reference member and/or a second reference member. The first reference member may include a first body having a first contact surface dimensioned according to a first patient-specific contour, which may be associated with a first non-articular surface of an anatomy. The first body may include a first passage having an opening along the first contact surface. The second reference member may be spaced apart from the first reference member. The second reference member may include a second body having a second contact surface dimensioned according to a second patient-specific contour, which may be associated with an articular surface of the anatomy. The second body may include a second passage established in the second body according to a placement of the first passage in the first body. A linking member may interconnect the first and second reference members.

A method of performing an orthopaedic procedure according to an implementation may include positioning a first patient-specific contact surface of a first reference member on a first non-articular surface of a bone of a patient. The method may include positioning a second patient-specific contact surface of a second reference member on an articular surface of the bone. The first and second reference members may be interconnected by a linking member. The method may include removing a portion of the bone to establish a bone graft in response to guiding a cutting instrument along a first resection surface of the first reference member. The method may include positioning the bone graft on the bone adjacent to the articular surface.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a planning system according to an implementation.

FIG. 2 discloses a planning system including a user interface according to another implementation.

FIG. 3 discloses an implementation of the user interface of FIG. 2 including different views of an anatomical model.

FIG. 4 discloses an implementation of the user interface of FIG. 2 including different views of an anatomical model and a graft model.

FIG. 5 discloses a portion of a guide model positioned relative to the anatomical model according to an implementation.

FIG. 6 discloses another portion of the guide model positioned relative to the anatomical model according to an implementation.

FIG. 7 discloses the guide model positioned relative to the anatomical model according to an implementation.

FIG. 8 discloses a perspective view of a guide according to an implementation.

FIG. 9 discloses a side view of the guide of FIG. 8.

FIG. 10 discloses an end view of the guide of FIG. 8.

FIG. 11 discloses another perspective view of the guide of FIG. 8.

FIG. 12 discloses a method of planning and performing an orthopaedic procedure according to an implementation.

FIG. 13 discloses a guide and one or more surgical instruments positioned relative to an anatomy according to an implementation.

FIG. 14 discloses another view of the guide of FIG. 13.

FIG. 15 discloses a perspective view of a bone graft positioned relative to the anatomy according to an implementation.

FIG. 16 discloses another view of the bone graft secured to the anatomy according to an implementation.

Like reference numbers and designations in the various drawings indicate like elements.

### DETAILED DESCRIPTION

This disclosure relates to surgical planning, systems and methods of repair. The planning systems described herein may be utilized for orthopaedic procedures and may be utilized to create, edit, execute and/or review surgical plans. The surgeon may utilize the planning systems pre-operatively, intra-operatively and/or post-operatively. The planning systems and method disclosed herein may include determining or inferring bone loss along an articular surface of a bone, such as a glenoid associated with a shoulder joint.

One or more dimensions associated with the bone loss may be determined and may relate to one or more patient-specific contours. The dimensions may be utilized to fabricate guide assemblies and other instrumentation, which may be used to form a bone graft and repair the articular surface with the bone graft. The instrumentation may include one or more features for precisely shaping the bone graft. The bone graft may be utilized in a Latarjet procedure to repair the articular surface. Correct fastener and/or graft/implant placement and trajectory during a Latarjet procedure may be difficult to control. Neurovascular aspects of the anatomy may need to be considered when performing the procedure. The instrumentation may be utilized in a coracoid osteotomy to harvest the bone graft. The planning system and instrumentation may be utilized to precisely position the bone graft, which may improve mobility and healing of the patient.

The disclosed guide may assist the surgeon in drilling in the correct location on the coracoid and may include a guide (e.g., resection) slot for performing a coracoid graft osteotomy. The guide may include a first reference member (e.g., coracoid guide component) and a second reference member (e.g., glenoid guide component), which may be spaced apart from each other. The guide may include a linking member (e.g., reference or strut) between the reference members. The reference members may be adapted to situate the guide in a patient-specific position and orientation. The reference members may include one or more patient-specific contact surfaces dimensioned to seat the guide on the anatomy according to a surgical plan. The reference members and/or another portion of the guide may include one or more guide features. The guide features may be adapted to guide instrumentation for harvesting the bone graft. The guide features may be adapted to guide instrumentation for removing tissue from the bone graft and/or preparing the anatomy for placement of the bone graft. The disclosed techniques may allow for the correct placement and trajectory when drilling passages (e.g., tunnels) in the glenoid, which may otherwise be performed free hand by the surgeon.

A guide for an orthopaedic procedure according to an implementation may include a first reference member and/or a second reference member. The first reference member may include a first body having a first contact surface dimensioned according to a first patient-specific contour, which may be associated with a first non-articular surface of an anatomy. The first body may include at least one guide feature dimensioned to guide a surgical instrument. The at least one guide feature may include a resection surface that may extend along a resection plane. The second reference member may be spaced apart from the first reference member. The second reference member may include a second body having a second contact surface dimensioned according to a second patient-specific contour, which may be associated with an articular surface of the anatomy. A linking member may interconnect the first and second reference members.

In any implementations, the at least one guide feature may include a resection slot that may extend along the resection surface.

In any implementations, the linking member may be dimensioned to span across a space between the articular surface and the first non-articular surface.

In any implementations, the first contact surface may extend along a first recess in the first body. The first recess may be dimensioned to receive a portion of bone associated with the first patient-specific contour.

In any implementations, the resection plane may intersect the first recess.

In any implementations, the at least one guide feature of the first body may include a first set of passages dimensioned to receive a surgical instrument. The second body may include a second set of passages dimensioned to receive a surgical instrument.

In any implementations, the second set of passages may be established in the second body according to a placement of the first set of passages in the first body.

In any implementations, the second contact surface may extend along a second recess in the second body. The second recess may be dimensioned to receive a portion of bone associated with the second patient-specific contour.

In any implementations, the second patient-specific contour may be associated with a second non-articular surface of the anatomy. The second recess may be dimensioned to capture a rim bounding the articular surface of the anatomy such that the second contact surface may abut the second non-articular surface.

In any implementations, the second body may include a passage. The passage may be dimensioned to guide an instrument to engage the anatomy below the rim.

In any implementations, the first and second reference members may be integrally formed with the linking member.

In any implementations, the second patient-specific contour may be associated with the articular surface of a glenoid.

In any implementations, the first patient-specific contour may be associated with a coracoid process.

A guide for an orthopaedic procedure according to an implementation may include a first reference member and/or a second reference member. The first reference member may include a first body having a first contact surface dimensioned according to a first patient-specific contour, which may be associated with a first non-articular surface of an anatomy. The first body may include a first passage having an opening along the first contact surface. The second reference member may be spaced apart from the first reference member. The second reference member may include a second body having a second contact surface dimensioned according to a second patient-specific contour, which may be associated with an articular surface of the anatomy. The second body may include a second passage established in the second body according to a placement of the first passage in the first body. A linking member may interconnect the first and second reference members.

In any implementations, the first and second passages may be dimensioned to receive a drill instrument.

In any implementations, the first passage may include a first set of passages. The second passage may include a second set of passages.

In any implementations, at least one of the first body and the second body may include a resection surface dimensioned to guide a cutting instrument.

In any implementations, the first and second reference members may be integrally formed with the linking member.

In any implementations, the second patient-specific contour may be associated with the articular surface of a glenoid.

In any implementations, the first patient-specific contour may be associated with a coracoid process.

A method of performing an orthopaedic procedure according to an implementation may include positioning a first patient-specific contact surface of a first reference member on a first non-articular surface of a bone of a patient. The method may include positioning a second patient-specific contact surface of a second reference member on an articular surface of the bone. The first and second reference members may be interconnected by a linking member. The method may include removing a portion of the bone to establish a bone graft in response to guiding a cutting instrument along a first resection surface of the first reference member. The method may include positioning the bone graft on the bone adjacent to the articular surface.

In any implementations, the method may include drilling a first set of holes in the portion of the bone through a first set of passages of the first reference member. The method may include drilling a second set of holes in the bone through a second set of passages of the second reference member. The step of positioning the bone graft may occur such that respective pairs of holes of the first and second sets of holes may be at least partially aligned with each other.

In any implementations, the method may include positioning a fastener through a respective pair of the holes to secure the bone graft to the bone.

In any implementations, the first and second reference members may be integrally formed with the linking member.

In any implementations, the method may include removing a portion of the bone graft to establish a first resection face. The method may include removing another portion of the bone to establish a second resection face, which may occur in response to guiding a cutting instrument along a second resection surface of the second reference member. The method may include positioning the first and second resection faces in abutment.

In any implementations, the articular surface may be associated with a glenoid of the patient.

In any implementations, the second patient-specific contact surface may be dimensioned to follow a second non-articular surface of the bone below a rim of the bone bounding the articular surface. The second contact surface may extend along a recess of the second reference member. The step of positioning the second contact surface may occur such that the rim may be captured by the recess.

In any implementations, the step of positioning the second patient-specific contact surface may occur such that the second patient-specific contact surface may be positioned on an anterior portion of the glenoid.

In any implementations, the second reference member may include a passage. The method may include guiding an instrument through the passage to form a hole in the second non-articular surface.

In any implementations, the first non-articular surface may be associated with a coracoid process of the patient.

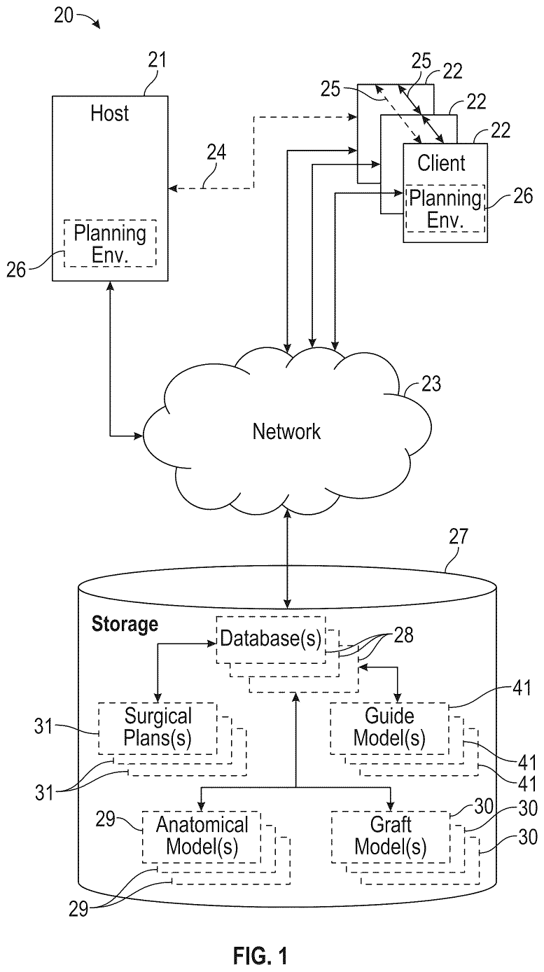

FIG. 1 discloses a planning system 20 according to an implementation. The system 20 may be used for planning various orthopaedic procedures, including pre-operatively, intra-operatively and/or post-operatively to create, edit, execute and/or review surgical plans for patients.

The system 20 may include a host computer 21 and one or more client computers 22. The host computer 21 may be operable to execute one or more software programs. In some implementations, the host computer 21 may be more than one computer jointly operable to process software instructions serially or in parallel.

The host computer 21 may be in communication with one or more networks such as a network 23 comprised of one or more computing devices. The network 23 may be a private local area network (LAN), a private wide area network (WAN), the Internet, or a mesh network.

The host computer 21 and each client computer 22 may include one or more of a computer processor, memory, storage means, network device and input and/or output devices and/or interfaces. The input devices may include a keyboard, mouse, or touch screen. The output devices may include monitors, speakers and printers. The memory may include UVPROM, EEPROM, FLASH, RAM, ROM, DVD, CD, a hard drive, or other computer readable medium which may store data and/or other information relating to any of the functionality disclosed herein. The host computer 21 and each client computer 22 may be a desktop computer, laptop computer, smart phone, tablet, or any other computing device. The interface may facilitate communication with the other systems and/or components of the network 23.

Each client computer 22 may be operable to access and locally and/or remotely execute a planning environment 26. The planning environment 26 may be a standalone software package or may be incorporated into another surgical tool. The planning environment 26 may be operable to provide a display or visualization of one or more bone models 29, graft models 30, guide (e.g., assembly) models 41 and related images via one or more graphical user interfaces (GUI). Each bone model, graft model and guide model, images and other information may be stored in one or more files or records according to a specified data structure.

Each client computer 22 may be operable to communicate with the host computer 21 directly via a direct client interface 24 or over the network 23. The client computers 22 may be operable to execute one or more software programs, such as various surgical tools. The planning environment 26 may be operable to communicate with the host computer 21 either over the network 23 or directly through the direct client interface 24. In another implementation, the client computers 22 may be operable to communicate with each other directly via a peer-to-peer interface 25.

The system 20 may include at least one storage system 27, which may be operable to store or otherwise provide data to other computing devices. The storage system 27 may be a storage area network device (SAN) operable to communicate with the host computer 21 and/or the client computers 22 over the network 23. In implementations, the storage system 27 may be incorporated within or directly coupled to the host computer 21 and/or client computers 22. The storage system 27 may be operable to store computer software instructions, data, database files and/or configuration information.

In implementations, the system 20 may be a client-server architecture operable to execute computer software on the host computer 21, which may be accessible by the client computers 22 using a thin client application or a web browser executing on the client computers 22. The host computer 21 may load the computer software instructions from local storage, or from the storage system 27, into memory and may execute the computer software using the computer processor(s).

The system 20 may include one or more databases 28. The databases 28 may be stored at a central location, such as the storage system 27. In another implementation, one or more databases 28 may be stored at the host computer 21 and/or may be a distributed database provided by one or more of the client computers 22. Each database 28 may be a relational database operable to associate one or more anatomical (e.g., bone) models 29, graft models 30 and/or guide models 41 to each other and/or a surgical plan 31. Each surgical plan 31 may be associated with a respective patient. Each anatomical model 29, graft model 30, guide model 41 and surgical plan 31 may be assigned a unique identifier or database entry. The database 28 may be operable to store data corresponding to the anatomical models 29, graft models 30, guide models 41 and surgical plans 31 in one or more database records or entries, and/or may be operable to link or otherwise associate one or more files corresponding to each respective anatomical model 29, graft model 30, guide model 41 and surgical plan 31. Anatomical models 29, graft models 30 and/or guide models 41 stored in the database(s) 28 may correspond to respective patient anatomies from prior and/or planned surgical cases, and may be arranged into one or more predefined categories such as sex, age, ethnicity, defect category and procedure type.

Each anatomical model 29 and graft model 30 may include information obtained from one or more medical devices or tools, such as a computerized tomography (CT), magnetic resonance imaging (MRI) machine and/or X-ray machine, that obtain one or more images of a patient. The anatomical model 29 and graft model 30 may include one or more digital images and/or coordinate information relating to an anatomy of the patient obtained or derived from the medical device(s). The anatomical model 29 may be associated with soft tissue (e.g., ligaments, tendons and/or skin) and/or one or more bones of the anatomy of a patient. The bones may be adjacent to each other and may be associated with a joint. The graft model 30 may include a portion of the geometry of a bone associated with the anatomical model 29 or may include separate and distinct geometry associated with another bone. Each guide model 41 may include coordinate information associated with a predefined design. The planning environment 26 may incorporate and/or interface with one or more modeling packages, such as a computer aided design (CAD) package, to render the models 29, 30, 41 as two-dimensional (2D) and/or three-dimensional (3D) volumes or constructs.

The predefined design may correspond to one or more components. The guide models 41 may correspond to instrumentation, devices and components of various shapes and sizes. Each guide may include one or more components that may be situated at a surgical site, such as guides and fasteners. Each guide model 41 may include two or more components that may be fixedly attached or otherwise secured to establish an guide. In implementations, the guide model 41 may correspond to a single component. Each anatomical model 29, graft model 30 and guide model 41 may correspond to 2D and/or 3D geometry, and may be utilized to generate a wireframe, mesh and/or solid construct in a display.

Each surgical plan 31 may be associated with one or more anatomical models 29, graft models 30 and/or guide models 41. The surgical plan 31 may include one or more revisions to the anatomical model 29, graft model 30 and/or guide model 41 and may include information relating to a position of a graft model 30 and/or guide model 41 relative to the original and/or revised anatomical model 29. The surgical plan 31 may include coordinate information relating to the revised anatomical model 29, graft model 30 and/or guide model 41 and a relative position of the models 29, 30, 41 in predefined data structure(s). Revisions to each model 29, 30, 41 and surgical plan 31 may be stored in the database 28 automatically and/or in response to user interaction with the system 20.

One or more surgeons, assistants and other clinical users may be provided with a planning environment 26 via the client computers 22 and may simultaneously access each anatomical model 29, graft model 30, guide model 41 and surgical plan 31 stored in the database(s) 28. Each user may interact with the planning environment 26 to create, view and/or modify various aspects of the surgical plan 31. Each client computer 22 may be operable to store local instances of the anatomical models 29, graft models 30, guide models 41 and/or surgical plans 31, which may be synchronized in real-time or periodically with the database(s) 28. The planning environment 26 may be a standalone software package executed on a client computer 22 or may be provided as one or more services executed on the host computer 21.

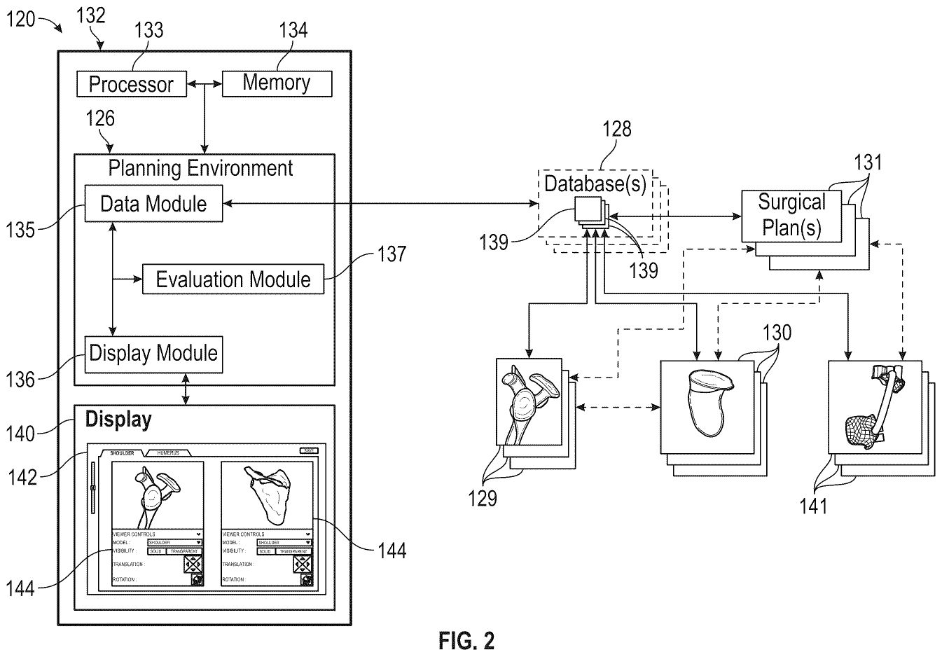

FIG. 2 discloses a planning system 120 for planning a surgical procedure. In this disclosure, like reference numerals designate like elements where appropriate and reference numerals with the addition of one-hundred or multiples thereof designate modified elements that are understood to incorporate the same features and benefits of the corresponding original elements. The system 120 may be utilized to plan and implement various orthopaedic and other surgical procedures, such as a Latarjet procedure or an arthroplasty to repair a joint. The system 120 may be utilized in planning a resection of one or more bones, including harvesting bones to establish bone grafts for repairing joints. The planning system 120 may be utilized in planning a Latarjet procedure to restore stability to a shoulder joint, which may address bone loss along a perimeter of a glenoid and an associated risk of dislocation and pain. The planning system 120 may be utilized in planning the harvesting or resection of a coracoid process of a patient for use in the Latarjet procedure. Although the planning systems and methods disclosed herein primarily refer to repair of a shoulder, the planning system 120 may be utilized in the repair of other locations of the anatomy and other surgical procedures including repair of other joints such as an ankle, wrist, hand, hip or knee, and including repair of fractures.

The system 120 may include various processing circuitry such as a computing device 132 including one or more processors 133 coupled to memory 134. The computing device 132 may include any of the computing devices disclosed herein, including the host computer 21 and/or client computer 22 of FIG. 1. The processor(s) 133 may be operable to collectively execute a planning environment 126 for creating, editing, executing and/or reviewing one or more surgical (e.g., pre-operative) plans 131 during pre-operative, intra-operative and/or post-operative phases of a surgery and for generating one or more models or designs that may be utilized to fabricate various guides and other assemblies, including any of the assemblies disclosed herein.

The planning environment 126 may include a data module 135, a display module 136 and an evaluation (e.g., spatial) module 137. Although four modules are shown, fewer or more than four modules may be utilized and/or one or more of the modules may be combined to provide the disclosed functionality.

The data module 135 may be operable to access, retrieve and/or store data and other information in the database(s) 128 corresponding to one or more anatomical (e.g., bone) model(s) 129, graft model(s) 130, guide (e.g., assembly) model(s) 141 and/or surgical plan(s) 131. The data and other information may be stored in one or more databases 128 as one or more records or entries 139. In implementations, the data and other information may be stored in one or more files that may be accessible by referencing one or more objects or memory locations referenced by the records or entries 139. The data module 135 may be operable to execute one or more system commands to communicate the data and other information.

The memory 134 may be operable to access, load, edit and/or store instances of one or more anatomical models 129, graft models 130, guide models 141 and/or surgical plans 131 in response to one or more commands from the data module 135. The data module 135 may be operable to cause the memory 134 to store a local instance of the anatomical model(s) 129, graft model(s) 130, guide model(s) 141 and/or surgical plan(s) 131 which may be synchronized with records 139 in the database(s) 128. The anatomical models 129 may be associated with any of the anatomy disclosed herein. The anatomical models 129 may be associated with one or more bones associated with a joint, such as a shoulder model associated with a shoulder of a patient. Other joints may include a hip joint of a patient associated with a pelvis and femur.

The display module 136 may be operable to display data and other information relating to one or more surgical plans 131 in at least one graphical user interface (GUI) 142, including the anatomical model(s) 129, graft model(s) 130, guide model(s) 141 and/or aspects of the surgical plan(s) 131. The computing device 132 may be coupled to a display device 140. The display module 136 may be operable to cause the display device 140 to display information in the user interface 142. The display module 136 may be operable to execute one or more system commands to display the data and other information. A surgeon or other clinical user may interact with the user interface 142 via the planning environment 126 to create, edit, execute and/or review one or more surgical plans 131.

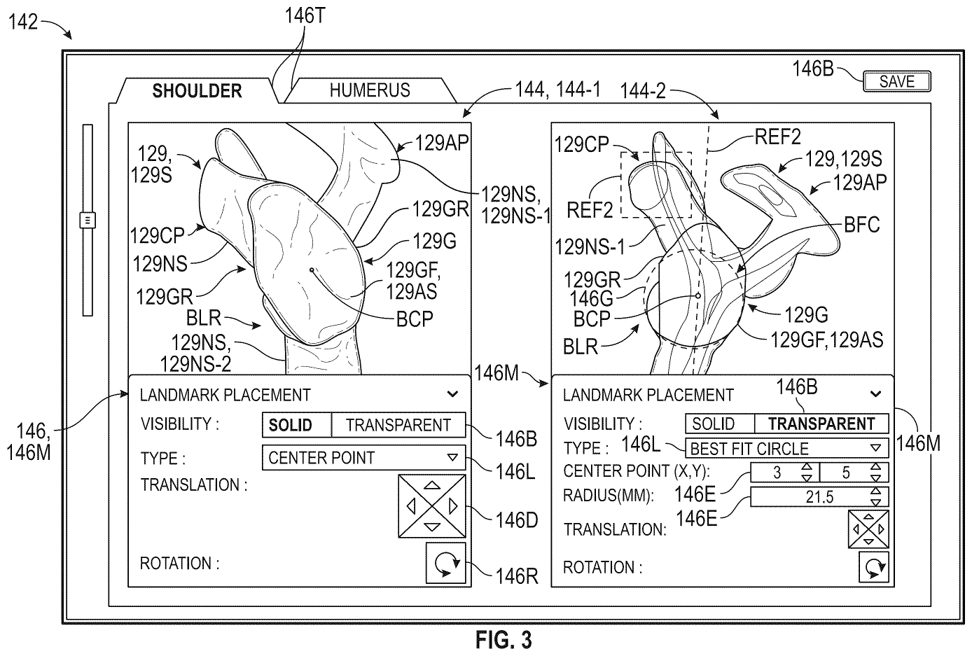

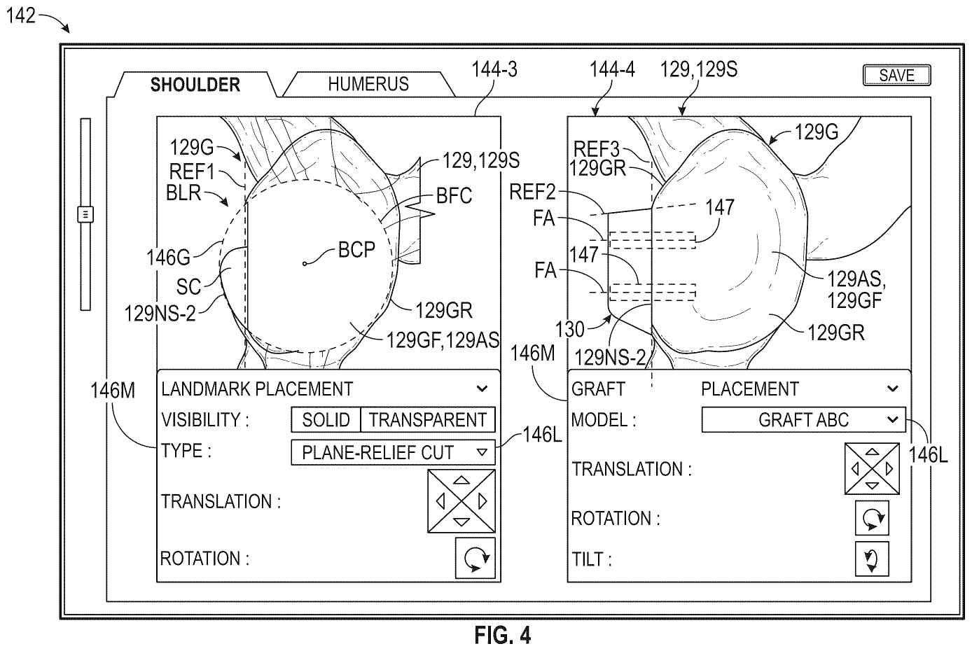

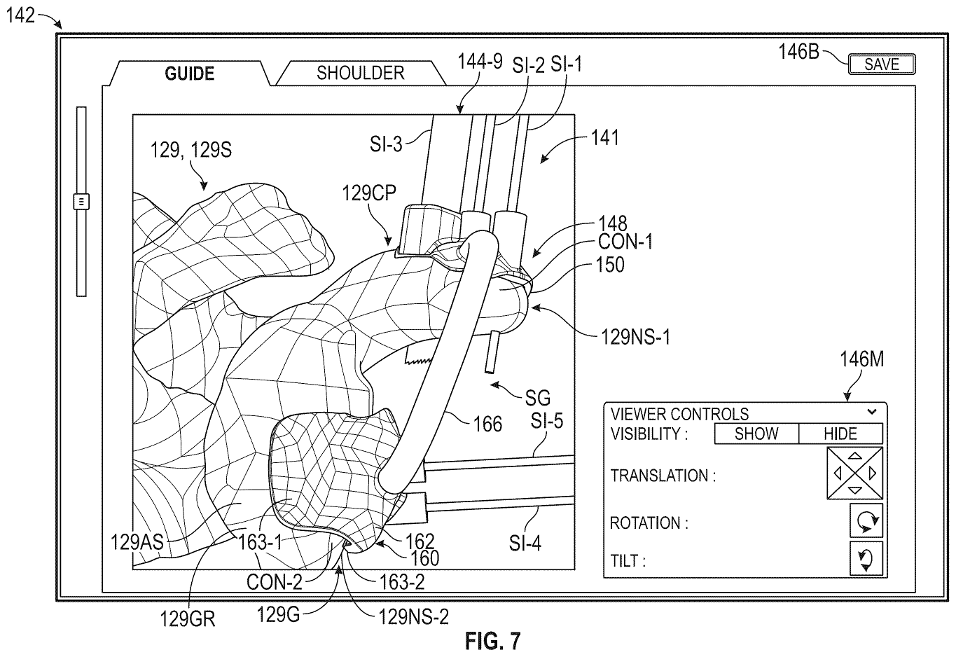

Referring to FIG. 3, with continuing reference to FIG. 2, the user interface 142 may include one or more display windows 144 and one or more objects 146. The objects 146 may include graphics such as menus, tabs and buttons accessible by user interaction, such as tabs 146T, buttons 146B, drop-down lists 146L, menus 146M, entry fields 146E, directional indicators 146D, 146R and graphics 146G. Geometric objects including selected anatomical model(s) 129, graft model(s) 130 (e.g., FIG. 4), and guide model(s) 141 (e.g., FIG. 7) and other information relating to the surgical plan 131 may be displayed in one or more of the display windows 144.

The display module 136 may be operable to display one or more selected anatomical models 129, graft models 130 and/or guide models 141 in the display windows 144. The selected anatomical model(s) 129, graft model(s) 130 and/or guide model(s) 141 may be selectively displayed and hidden (e.g., toggled) in one or more of the display windows 144 in response to user interaction with the user interface 142, which may provide the surgeon with enhanced flexibility in determining and/or reviewing aspects of the surgical plan 131.

The data module 135 may be operable to access the anatomical model 129 from the database 128, which may occur automatically or in response to user interaction with the user interface 142. The data module 135 may be operable to store an instance of the selected anatomical model 129 in the memory 134. The display module 136 may be operable to display the selected anatomical model 129 in at least one of the display windows 144 of the user interface 142. The anatomical model 129 may be associated with a joint, including a shoulder or hip joint. In implementations, the anatomical model 129 may be a shoulder model 129S associated with a shoulder establishing a shoulder joint of a patient. The shoulder model 129S may include a glenoid 129G including a glenoid face 129GF and glenoid rim 129GR, an acromion process 129AP and a coracoid process 129CP.

The display windows 144 may include first and second display windows 144-1, 144-2. Although a particular number of display windows 144 are disclosed in FIG. 3, the user interface 142 may include any number of display windows 144 in accordance with the teachings disclosed herein, and aspects of the display windows 144 may be combined or separated. The display windows 144 may be operable to display various views of the anatomical model(s) 129. The display windows 144 may be operable to display a two-dimensional (2D) and/or three-dimensional (3D) representation of the selected anatomical model(s) 129. The first display window 144-1 may be operable to display the anatomical model 129 at a first orientation, such as a lateral view of the glenoid face 129GF. The second window 144-2 may be operable to display the anatomical model 129 at a second orientation, which may be the same or may differ from the first orientation. The surgeon or assistant may interact with the menus 146M, directly with the display windows 144-1, 144-2, or with another portion of the user interface 142 to move the selected anatomical model 129 in 2D space (e.g., up, down, left, right) and/or 3D space (e.g., rotation, tilt, zoom), which may occur in response to interaction with the directional indicators 146D, 146R. The display module 136 may be operable to cause the display of a transparency of the anatomical model 129 in the display window 144 of the user interface 142, such as the second display window 144-2, which may occur automatically or in response to selection of one of the buttons 146B of the associated menu 146M.

The selected anatomical model 129 may include one or more articular surfaces 129AS and/or non-articular surfaces 129NS. The articular surface 129AS may be associated with an articular surface of a bone having an amount of bone loss or erosion. The bone loss may reside in a bone loss region BLR. The bone loss region BLR may reside along, or may otherwise be adjacent to, a perimeter of the articular surface 129AS, such as the glenoid rim 129GR. A graphic 146G associated with the bone loss region BLR may be displayed in the display window 144-2.

The system 120 may be operable to determine or otherwise approximate an amount of bone loss associated with an articular surface of a bone corresponding to the selected anatomical model 129, such as bone loss associated with the bone loss region BLR. The perimeter of the glenoid face 129GF may be established by the glenoid rim 129GR and the bone loss region BLR. The first display window 144-1 may be operable to display a solid, lateral view of the articular surface 129AS, such as the glenoid face 129GF and the bone loss region BLR. The evaluation module 137 may be operable to determine one or more dimensions associated with the bone loss region BLR.

Various techniques may be utilized to determine (e.g., approximate) the bone loss region BLR. In implementations, the evaluation module 137 may be operable to establish a best fit circle BFC along the articular surface 129AS, such as the glenoid face 129GF of the shoulder model 129S, which may be displayed in the second display window 144-2. A point BCP may be specified or otherwise established. The point BCP may correspond to a center of the best fit circle BFC. The evaluation module 137 may be operable to cause the display module 136 to display the best fit circle BFC and point BCP along the articular surface 129AS. In implementations, the user may interact with the display window 144-2 and/or another portion of the user interface 142 to set a position of the point BCP. The user may interact with the menu 146M or another portion of the user interface 142 to set or adjust a radius associated with the point BCP to establish the best fit circle BFC. The bone loss region BLR may be established between the perimeter of the best fit circle BFC and the perimeter of the articular surface 129AS, such as the glenoid rim 129GR.

Referring to FIGS. 3 and 4, with continuing reference to FIG. 2, the display windows 144 may include third display windows 144-3, 144-4 (FIG. 4). The user may interact with the menu 146M associated with the display window(s) 144, directly with the display window(s) 144 and/or another portion of the user interface 142 to establish one or more reference planes associated with a simulated relief (e.g., bevel) cut, such as a first reference plane REF1 and/or a second reference plane REF2 (FIG. 2). The evaluation module 137 may be operable to adjust a geometry of the bone loss region BLR based on the reference plane REF1. The evaluation module 137 may be operable to determine one or more dimensions with respect to the reference planes REF1 and/or REF2. The display windows 144-1, 144-2 and/or 144-3, 144-4 may be linked to each other and may be operable to display different orientations of the anatomical model 129, such as the coracoid process 129CP of the anatomical model 129 associated with the bone loss region BLR, or another bone model 129 associated with another bone or joint of the patient anatomy.

The evaluation model 137 may be operable for evaluating one or more regions of the anatomy for harvesting a bone graft (e.g., fragment). The bone graft may be an autograft associated with the same patient as the anatomical model 129 associated with the bone loss region BLR or may be an allograft associated with another person for transplanting into the patient. The evaluation module 137 may be operable to cause the display module 136 to display the reference plane REF2 in the display window 144-2 relative to the coracoid process 129CP of the selected anatomical model 129 (FIG. 3). The user may interact with the menu 146M or another portion of the user interface 142 to select a graft model 130 (e.g., FIG. 4), such as the graft model 130 associated with the simulated relief cut established by the reference plane REF2.

The user may interact with the user interface 142 to modify a geometry of the selected graft model 130. The user may interact with the menu 146M associated with the display window 144-4, directly with the display windows 144-4 and/or another portion of the user interface 142 to prepare (e.g., shape) a geometry of the graft model 130. Preparing the graft model 130 may include establishing one or more reference planes associated with a simulated relief cut, such as a third reference plane REF3. The reference plane REF3 may be associated with sizing (e.g., cutting or trimming) a bone graft associated with the graft model 130. The evaluation module 137 may be operable to cause the display module 136 to display the reference plane REF3 in the display window 144-4.

The surgeon or assistant may interact with the menu 146M associated with the display window 144-4 and/or another portion of the user interface 142 to approve of a volume associated with the bone graft model 130. The graft model 130 may be associated with the coracoid process 129CP of the shoulder model 129S. The evaluation module 137 may be operable to generate the graft model 130 and may be operable to cause the data module 135 to store the graft model 130 to memory 134 and/or the database 128. The graft model 130 may be associated with the determined dimensions and/or modifications, including one or more cuts associated with the reference plane(s) REF2, REF3. The graft model 130 may be established in response to setting the reference plane REF2 and/or REF3, which may simulate a resection of the associated bone. The evaluation module 137 may be operable to cause changes to the graft model 130 to be stored in the memory 134 and/or database 128, including the reference planes REF2, REF3 associated with the simulated relief cuts.

The surgeon or assistant may interact with the menu 146M associated with the display window 144-4, directly with the display window 144-4 and/or another portion of the user interface 142 to position the graft model 130 with respect to an (e.g., selected) anatomical model 129. The user may interact with the user interface 142 to move the selected graft model 130 relative to the anatomical model 129. The user may interact with the user interface 142 to move the graft model 130 into abutment with a surface of the anatomical model 129, such as a non-articular surface 129NAS. The non-articular surface 129NAS may be adjacent to an articular surface 129AS of the anatomical model 129, such an anterior surface of the glenoid 129G.

The evaluation module 137 may be operable to position the selected anatomical and graft models 129, 130 into contact with each other at a specified or defined position and orientation, which may be according to user interaction with the display windows 144, menu 146M, and/or other objects 146 of the user interface 142. The display module 136 may be operable to display the anatomical model 129 and graft model 130 relative to each other.

The user may interact with the user interface 142 to set a position and orientation of one or more fastener axes FA. The fastener axis FA may be associated with a trajectory of a respective fastener for securing a bone graft associated with the graft model 130. The user may interact with the menu 146M associated with the display window 144-4, directly with the display window 144-4, or with another portion of the user interface 142 to set a position and orientation of the fastener axis FA. The fastener axis FA may be associated with a respective fastener model 147, which may be stored in one or more records 139 of the database 128. The fastener models 147 may be associated with respective fasteners, such as pins, nails, cannulated and non-cannulated (e.g., compression) screws. Other fastening techniques may be utilized, such as soft suture fixation. The fastener model(s) 147 may be utilized to simulate securing a bone graft with one or more fasteners at respective insertion points along the anatomical model 129 established by the respective fastener axes FA. The insertion point may be established along the non-articular surface 129NAS of the anatomical model 129.

The surgeon or assistant may interact with the user interface 142 to approve the surgical plan 131, including a geometry, placement, revisions (e.g., cuts and drill holes), fastener positions, and/or other parameters associated with the selected anatomical model 129 and graft model 130. The data module 135 may be operable to store the approved surgical plan 131 and any subsequent changes in the memory 134 and/or database 128.

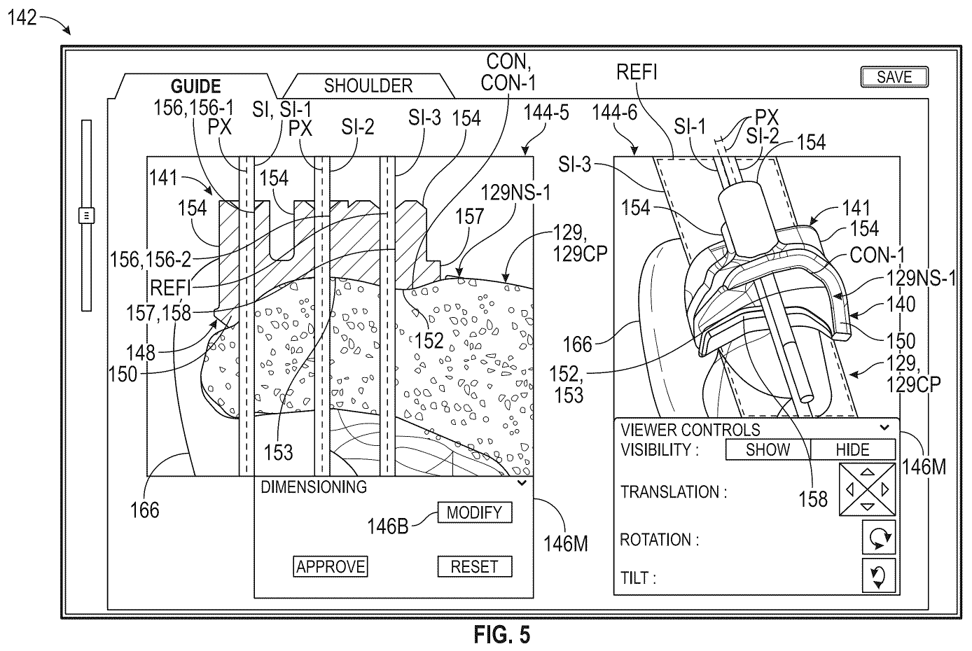

Referring to FIG. 5, with continuing reference to FIG. 2, the system 120 may be operable to establish one or more dimensions or settings associated with instrumentation. The instrumentation may be utilized in preparing a patient anatomy, harvesting a bone graft and/or placing a bone graft in an orthopaedic procedure, including any of the procedures disclosed herein such as a Latarjet procedure. The evaluation module 137 may be operable to generate one or more guide (e.g., assembly) models 141 associated with the dimensions or settings. The guide model 141 may be patient-specific or may be patient-generic. The guide model 141 may be associated with one or more patient-specific and/or reusable features (e.g., components).

The display windows 144 may include fifth and sixth display windows 144-5, 144-6. The user may interact with the menu 146M, directly with the display window(s) 144-5, 144-6 and/or another portion of the user interface 142 to specify, modify and/or otherwise establish a geometry of the guide model 141, including any dimensions and/or features.

The guide model 141 may include feature(s) dimensioned to reference one or more surfaces associated with an anatomy of a patient. The guide model 141 may be dimensioned with respect to one or more patient-specific contours CON, which may be associated with a respective anatomical model 129, graft model 130 and/or surgical plan 131 for a patient. The patient-specific contours CON may include a first patient-specific contour CON-1, which may be associated with a (e.g., first) non-articular surface 129NS-1 of the anatomy. The first contour CON-1 may be associated with a selected graft model 130, which may be associated with a portion of bone harvested from the anatomy to establish a graft.

The guide model 141 may include a first reference member (e.g., coracoid guide component) 148, which may be dimensioned to seat or otherwise position the guide model 141 on the anatomy, including the patient-specific contour(s) CON of the anatomical model 129. The first reference member 148 may include a first body 150. The first body 150 may include a first contact surface 152. The first contact surface 152 may have a patient-specific contour or may have one or more geometric surfaces, which may be dimensioned to position the reference member 148 in a patient-specific manner relative to the anatomy. The first contact surface 152 may be dimensioned according to the first patient-specific contour CON-1 associated with the first non-articular surface 129NS-1 of the anatomy. The first contact surface 152 may be dimensioned to follow the first patient-specific contour CON-1. Various techniques may be utilized to establish the first contact surface 152. The user may interact with the menu 146M, directly with the display window(s) 144-5, 144-6 and/or another portion of the user interface 142 to position the first reference member 148 relative to the anatomical model 129, such as the coracoid process 129CP. A portion of the first reference member 148 overlapping with a volume of the anatomical model 129 may be subtracted from the volume of the first reference member 148 to establish the first contact surface 152. In implementations, the first contact surface 152 may extend along a first recess 153 in the first body 150. The first recess 153 may be dimensioned to receive a portion of bone associated with the first patient-specific contour CON-1. The portion of bone may be associated with the graft model 130 (e.g., FIG. 4).

The user may interact with the user interface 142 to modify aspects of the guide model 141, including the first reference member 148. In implementations, the user may interact with a button 146B to edit the generated reference member 148 in another portion of the planning environment 126, or in a separate CAD tool. The user may edit the generated reference member 148 to incorporate various features, including recesses, grooves, passages, openings and/or flanges. The evaluation module 137 may be operable to cause the data module 135 to store the reference member 148, including revisions and other aspects of the generated guide model 141, in the memory 134 and/or a record 139 in the database 128. Edits to the guide model 141 may be saved to the memory 134 or to the database 128 as one or more records 139.

One or more guide features 154 may be established in the guide model 141, including the first reference member 148. The guide feature(s) 154 may include various geometries and may be dimensioned to guide various surgical instruments SI, including guide elements such as Kirschner wires and guide pins, burrs, reamers, drill bits and saw blades. In implementations, the surgical instruments SI may include one or more drill bits SI-1, SI-2 and/or saw blades SI-3. The user may interact with the menu 146M, directly with the display window(s) 144, or another portion of the user interface 142 to set a position, orientation and/or dimension of each of the guide features 154 associated with the guide model 141. The guide features 154 may include one or more guide (e.g., drill) passages 156, which may be dimensioned to extend along respective passage axes PX. The guide features 154 may include one or more (e.g., resection) surfaces 157, which may be dimensioned to resect a portion of bone. The resected portion of bone may be associated with the graft model 130. The resection surface(s) 157 may be established by one or more guide (e.g., resection or instrument) planes REFI. The resection surface 157 may be dimensioned to extend along the respective reference plane REFI. In implementations, the guide features 154 may include a slot 158 that may establish respective resection surface(s) 157. In implementations, a side wall of the first body 150 may be dimensioned to establish a respective resection surface 157 (e.g., display window 144-5). The resection surface 157 may be established such that the reference plane REFI may not intersect the passage axes PX, at least within a volume of the anatomical model 129.

The user may interact with the menu 146M, directly with the display window(s) 144-5, 144-6 and/or another portion of the user interface 142 to set a trajectory of the passage axes PX and/or reference plane REFI. A bone graft may be established in response to guiding a saw blade along the respective reference plane REFI to sever a portion of the bone from the anatomy. The guide model 141 may be established such that the reference plane REFI may be adjacent to a tip of the coracoid process 129CP. Severing the bone may include severing the tip of the coracoid process.

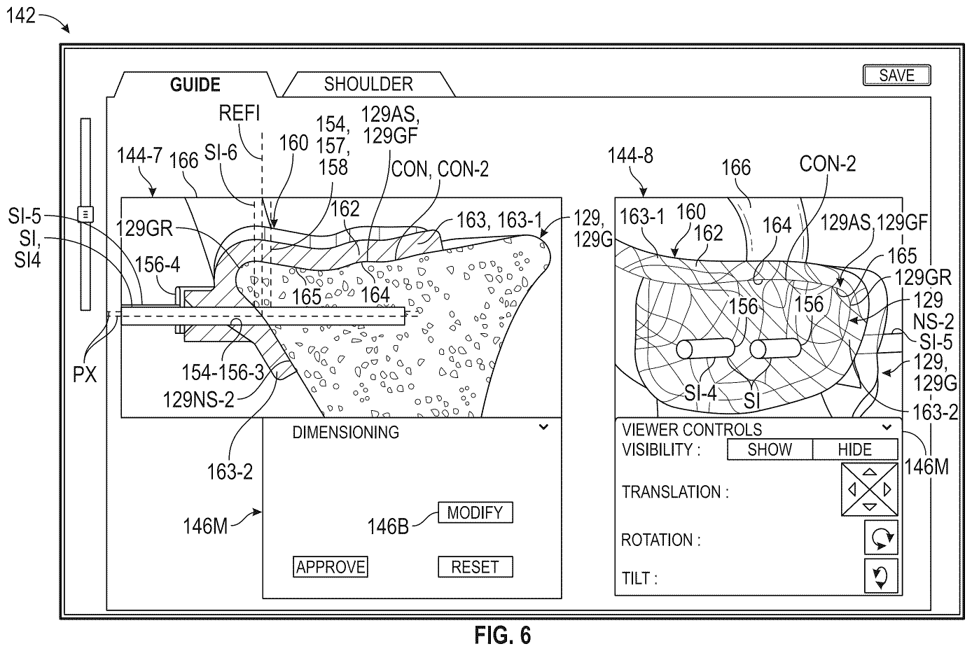

Referring to FIG. 6, with continuing reference to FIGS. 2 and 4-5, the guide model 141 may include one or more features dimensioned to establish a position of the graft model 130 and associated guide relative to articular and/or non-articular surfaces of a bone associated with the selected anatomical model 129, such as the glenoid face 129GF of the glenoid 129G or another articular surface 129AS. The display windows 144 may include seventh and eighth display windows 144-7, 144-8.

The guide model 141 may include a second reference member (e.g., glenoid guide component) 160, which may be dimensioned to seat or otherwise position the guide model 141 on the anatomy, including patient-specific contour(s) CON of the anatomical model 129. The patient-specific contours CON may include a second patient-specific contour CON-2. The second patient-specific contour CON-2 may be associated with the articular surface 129AS and/or a (e.g., second) non-articular surface 129NS-2 of the anatomy. The second non-articular surface 129NS-2 may extend from, or may otherwise be adjacent to, the articular surface 129AS. The first and second non-articular surfaces 129NS-1, 129NS-2 may be spaced apart from each other.

The second reference member 160 may incorporate any of the features of the first reference member 148, and vice versa. The second reference member 160 may include a second body 162. The second body 162 may include a second contact surface 164. The second contact surface 164 may have a patient-specific contour or may have one or more geometric surfaces, which may be dimensioned to position the reference member 148 in a patient-specific manner relative to the anatomy. The second contact surface 164 may be dimensioned according to the second patient-specific contour CON-2 associated with the articular surface 129AS and/or the second non-articular surface 129NS-2 of the anatomy. The second contact surface 164 may be dimensioned to follow the second patient-specific contour CON-2. Various techniques may be utilized to establish the second contact surface 164. The user may interact with the menu(s) 146M, directly with the display window(s) 144-7, 144-8 and/or another portion of the user interface 142 to position the second reference member 160 relative to the anatomical model 129, such as the glenoid 129G. A portion of the second reference member 160 overlapping with a volume of the anatomical model 129 may be subtracted from the volume of the second reference member 160 to establish the second contact surface 164. In implementations, the second contact surface 164 may extend along a second recess 165 in the second body 162. The second recess 165 may be dimensioned to receive a portion of bone associated with the second patient-specific contour CON-2. The portion of bone may be associated with the glenoid, such as the rim 129GR of the glenoid 129G.

The system 120 may be operable to establish one or more flanges 163 of the selected guide model 141. The second body 162 may include a first flange 163-1 and/or a second flange 163-2. The first flange 163-1 may be dimensioned to follow the articular surface 129AS of the bone model 129. The second flange 163-2 may be dimensioned to follow the second non-articular surface 129NS-2 of the anatomical model 129. The evaluation module 137 may be operable to establish the flange(s) 163 utilizing various techniques, such as an extrusion operation.

The user may interact with the user interface 142 to modify aspects of the guide model 141, including the second reference member 160. In implementations, the user may interact with a button 146B to edit the generated reference member 160 in another portion of the planning environment 126, or in a separate CAD tool. The user may edit the generated reference member 160 to incorporate various features, including recesses, grooves, passages, openings and/or flanges. The evaluation module 137 may be operable to cause the data module 135 to store the reference member 160, including revisions and other aspects of the generated guide model 141, in the memory 134 and/or a record 139 in the database 128. Edits to the guide model 141 may be saved to the memory 134 or to the database 128 as one or more records 139.

One or more guide features 154 may be established in the second reference member 160. In implementations, the surgical instruments SI may include one or more drill bits SI-4, SI-5 and/or saw blades SI-6. The guide features 154 may include one or more guide passages 156 and/or one or more guide (e.g., resection) slots 158. The user may interact with the menu(s) 146M, directly with the display window(s) 144-7, 144-8 and/or another portion of the user interface 142 to set a trajectory of the passage axes PX and/or reference plane REFI associated with the respective instruments SI. A portion of bone may be removed along, or otherwise adjacent to, the glenoid rim 129GR and/or another portion of the anatomy in response to guiding the saw blade SI-6 along the respective reference plane REFI. In implementations, the resection slot 158 may be omitted (e.g., display window 144-8).

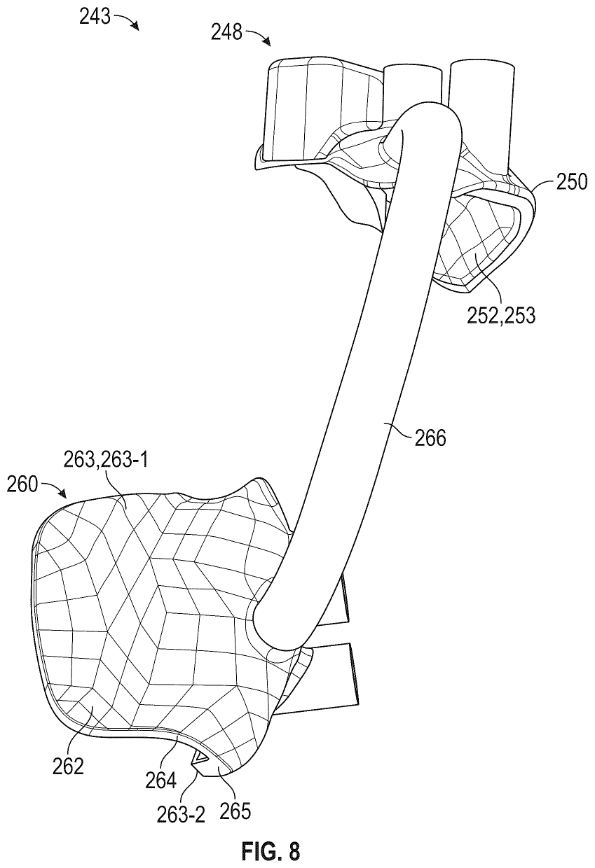

Referring to FIG. 7, with continuing reference to FIGS. 2 and 5-6, the display windows 144 may include a ninth display window 144-9. The display window 144-9 may be operable to display an (e.g., selected or generated) guide model 141 in various orientations and/or positions. The guide model 141 may be associated with a guide (e.g., guide 243 of FIGS. 8-11). The guide may be utilized for a surgical procedure, including any of the orthopaedic procedures disclosed herein. The display module 136 may be operable to display the resultant guide model 141 in various positions and/or orientations relative to a selected anatomical model 129 in the display window 144-9.

The guide model 141 may include a linking member 166. The linking member 166 may be dimensioned to set a (e.g., fixed) position of the first and second reference members 148, 160 relative to each other. The linking member 166 may be dimensioned to space apart the reference members 148, 160. Each of the reference members 148, 160 may have a unitary construction. The reference members 148, 160 may be integrally formed with each other and/or the linking member 166. In other implementations, the linking member 166 may be adjustable to vary a position and/or orientation of the reference members 148, 160 relative to each other. The linking member 166 may be dimensioned to span across a space (e.g., gap) SG between the first non-articular surface 129NS-1 and the articular surface 129AS and/or second non-articular surface 129NS-2.

Various techniques may be utilized to establish the linking member 166. In implementations, an object may be extruded along a spline between a first point on the first reference member 148 and a second point on the second reference member 160. The linking member 166 may be generated automatically and/or in response to user interaction with the menu 146M, directly with the display window 144-9, and/or another portion of the user interface 142. The user may set, adjust or otherwise specify the points on the reference members 148, 160 and/or points along the spline in response to user interaction with the user interface 142.

The first body 150 may include a first set of passages 156 (e.g., 156-1, 156-2 of FIG. 5) dimensioned to receive a surgical instrument SI. The second body 162 may include a second set of passages 156 (e.g., 156-3, 156-4 of FIG. 6) dimensioned to receive a surgical instrument SI. The second set of passages 156 may be established in the second body 162 according to a placement of the first set of passages 156 in the first body 150. A relative spacing and orientation between the passages 156-3, 156-4 may be the substantially the same as between the passages 156-1, 156-2. For the purposes of this disclosure, the terms “substantially,” “approximately” and “about” mean ±5 percent of the stated value or relationship unless otherwise stated. The passages 156 and respective passage axes PX may be established such that a graft associated with the graft model 130 may be situated along the anatomical model 129 at a (e.g., specified or predetermined) position, including at a height or offset from the articular surface 129AS of the associated anatomical model 129.

The planning environment 126 may be operable to save the guide model 141 to the memory 134 and/or database 128 automatically and/or in response to user interaction. The planning environment 126 may be operable to cause a physical guide (e.g., assembly) associated with the guide model 141 and/or an associated configuration (e.g., file) to be generated. The user may interact with one or more buttons 146B or another portion of the user interface 142 to save the guide model 141 and/or cause a physical guide and/or configuration associated with the guide model 141 to be generated. The generating may include causing the physical guide to be fabricated according to dimensions and geometry specified in the guide model 141. The physical guide may include one or more patient-specific surfaces and other features associated with the respective surgical plan 131.

FIGS. 8-11 disclose a guide (e.g., assembly) 243 according to an implementation. The guide 243 may be utilized to perform any of the surgical procedures disclosed herein, including orthopaedic procedures such as a Latarjet procedure. The guide 243 may be designed, fabricated or otherwise formed utilizing any of the techniques disclosed herein, including the planning system 120. The guide 243 may be dimensioned according to a respective guide model 141 (e.g., FIGS. 2 and 7). The guide 243 may incorporate any of the features of the guide model 141 disclosed herein.

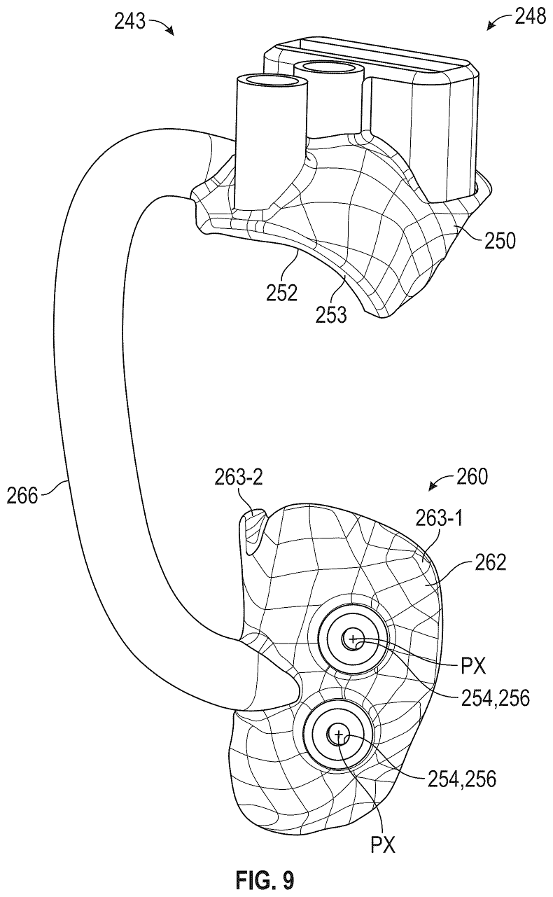

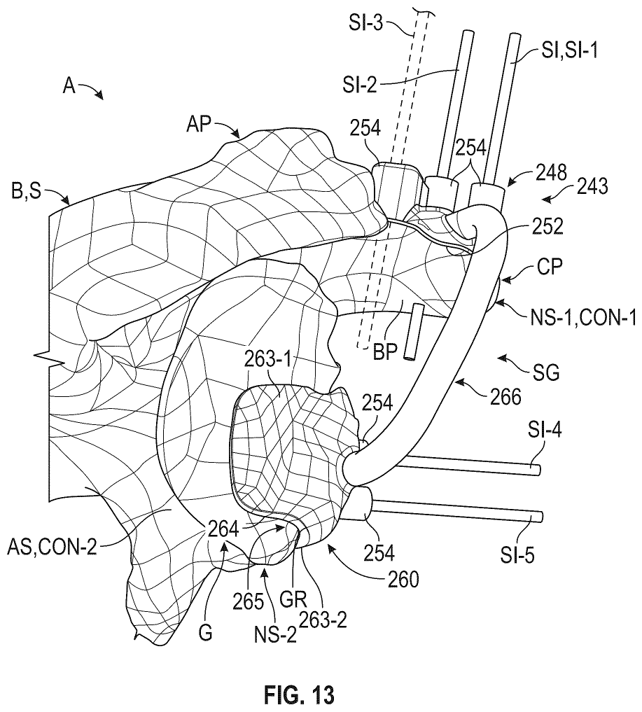

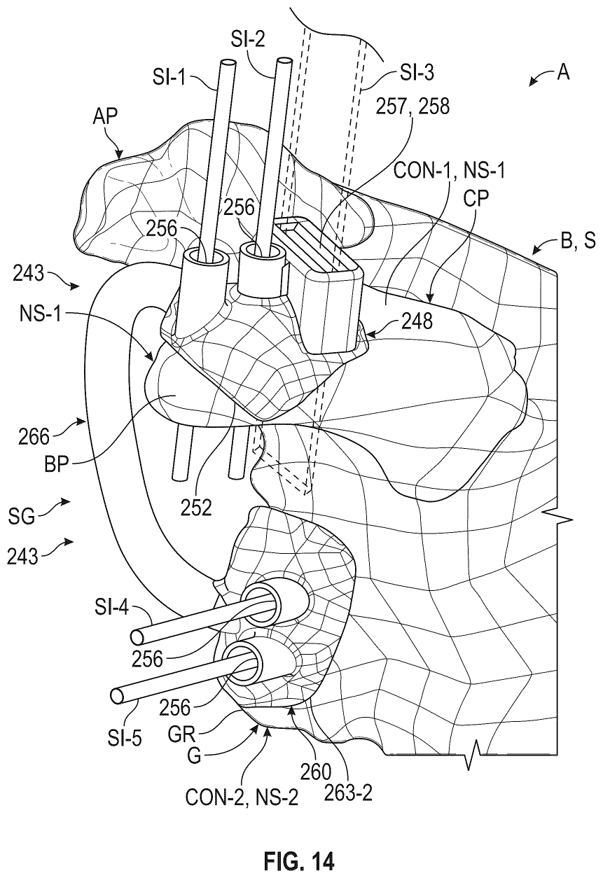

Referring to FIGS. 8-9, with reference to FIGS. 2, 7 and 13-14, the guide 243 may include a first reference member (e.g., coracoid guide component) 248 and/or a second reference member (glenoid guide component) 260. The first and/or second reference members 248, 260 may be dimensioned to seat or otherwise place the guide 243 on the anatomy in a patient-specific position and/or orientation, which may be specified in a respective surgical plan 131 (FIG. 2). The first reference member 248 may include a first body 250 having a first contact surface 252. The first contact surface 252 may be a single contiguous surface or may include two or more discontinuous surfaces. The first contact surface 252 may be dimensioned according to a first patient-specific contour CON-1 associated with a first non-articular surface NS-1 of an anatomy A of a patient (e.g., FIGS. 13-14).

The first contact surface 252 may extend along a first recess 253 in the first body 250. The first recess 253 may be dimensioned to receive a portion of bone B associated with the first patient-specific contour CON-1 (e.g., FIGS. 13-14). In implementations, the first patient-specific contour CON-1 may be associated with a portion of a scapula S, such as the coracoid process CP. In other implementations, the first patient-specific contour CON-1 may be associated with an acromion process AP of the scapula S. The first contact surface 252 may be dimensioned to follow the first patient-specific contour CON-1 along the first non-articular surface NS-1 of the anatomy A.

The second reference member 260 may include a second body 262 having a second contact surface 264. The second contact surface 264 may be dimensioned according to a second patient-specific contour CON-2 associated with an articular surface AS of the anatomy A (e.g., FIGS. 13-14). The second patient-specific contour CON-2 may be associated with the articular surface AS of a glenoid G. The second contact surface 264 may be a single contiguous surface or may include two or more discontinuous surfaces. The second contact surface 264 may extend along a second recess 265 in the second body 262. The second recess 265 may be dimensioned to receive a portion of bone B associated with the second patient-specific contour CON-2. In implementations, the second patient-specific contour CON-2 may be associated with a second non-articular surface NS-2 of the anatomy A, such as a non-articular surface NS-2 of the glenoid G, which may be below (e.g., medial to) the glenoid rim GR. The second contact surface 264 may be dimensioned to follow the second patient-specific contour CON-2 along the articular surface AS and/or second non-articular surface NS-2 of the anatomy A. The second recess 265 may be dimensioned to capture the rim GR bounding the articular surface AS of the anatomy A such that the second contact surface 264 may abut the second non-articular surface NS-2. The second body 262 may include one or more flanges 263, including a first flange 263-1 and/or a second flange 263-2. The first flange 263-1 may be dimensioned to follow the articular surface AS of the bone B. The second flange 263-2 may be dimensioned to follow the second non-articular surface NS-2 of the bone B.

The first and second reference members 248, 260 may be spaced apart from each other. The guide 243 may include a linking member 266. The linking member 266 may interconnect the members 248, 260. The reference members 248, 260 may be integrally formed with each other and/or the linking member 266. In other implementations, the reference members 248, 260 and linking member 266 may be separate and distinct components that may be fixedly attached or otherwise secured to each other. The linking member 266 may serve as a handle for manipulating the guide 243. In other implementations, the guide 243 may include a separate handle.

Referring to FIGS. 9-11, with continuing reference to FIGS. 2, 7-8 and 13-14, the first body 250 and/or second body 262 of the guide 243 may include one or more guide features 254, including any of the guide features disclosed herein. The guide feature(s) 254 may include various geometries and may be dimensioned to guide various surgical instruments, including any of the instruments disclosed herein. The guide features 254 may be dimensioned according to any of the techniques disclosed herein.

The guide features 254 may include one or more guide passages 256, which may be dimensioned to extend along respective passage axes PX (e.g., FIG. 9). The passages 256 may be dimensioned to guide one or more surgical instruments, such as a drill instrument (e.g., drill bit) or guide element, to form one or more holes in a bone B of the anatomy A. The holes may be dimensioned to receive one or more fasteners for securing a bone graft. The first body 250 and/or second body 262 may include one or more passages 256. In implementations, the first body 250 may include a first set of passages 256 dimensioned to receive a surgical instrument. The first set of passages 256 may have openings along the first contact surface 252 (e.g., FIG. 11). The second body 262 may include a second set of passages 256 dimensioned to receive a surgical instrument. The second set of passages 256 may have openings along the second contact surface 264 (e.g., FIG. 11). The second set of second passages 256 may be established in the second body 262 according to a placement of the first set of passages 256 in the first body 250. A passage 256 may be dimensioned to guide a surgical instrument to engage the anatomy below the rim of the articular surface AS, such as the glenoid rim GR.

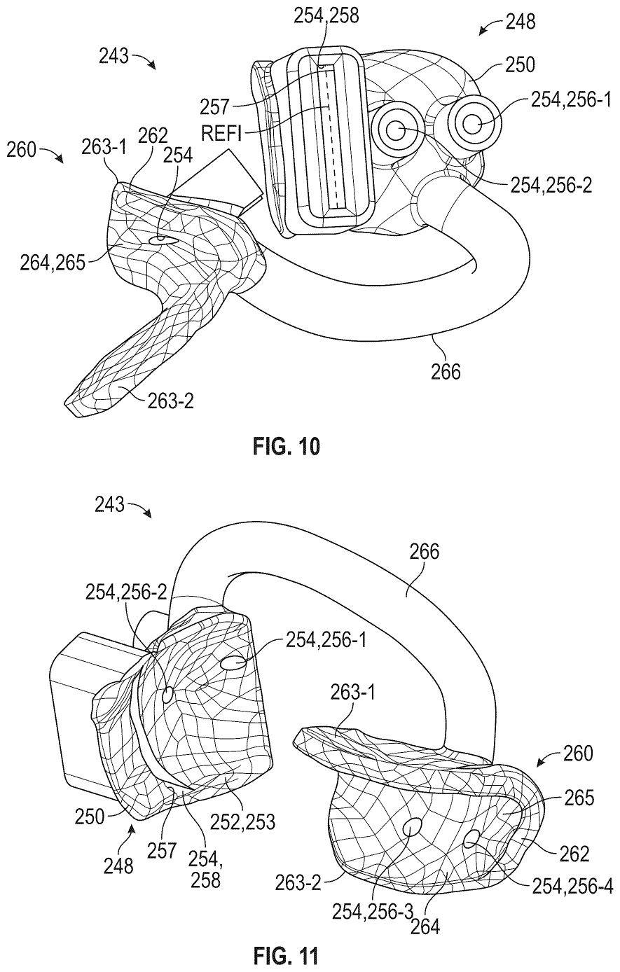

The guide features 254 may include one or more (e.g., resection) surfaces 257 (e.g., FIG. 10), which may be dimensioned to guide a cutting instrument to resect a portion of bone. The first body 250 may include one or more resection surfaces 257. The resection surface(s) 257 may be established by one or more (e.g., resection or instrument) planes REFI. The resection surface 257 may be dimensioned to extend along the respective reference plane REFI. In implementations, the guide features 254 may include a (e.g., resection) slot 258 that may establish respective resection surface(s) 257. The first body 250 may include the resection slot 258. The resection slot 258 may extend along a reference plane REFI that may intersect the first recess 253. The resection surface 257 may be dimensioned to guide a surgical instrument, such as a saw blade or other cutting instrument, to sever (e.g., harvest) a portion of bone B to establish a bone graft, such as a portion of the coracoid process CP (e.g., FIGS. 13-14).

The surgeon may guide a drill bit through the first set of passages 256 to form holes in a portion of the bone B, which may occur prior to severing the portion of bone B from the anatomy A to establish a bone graft. The surgeon may guide a drill bit through the second set of passages 256 to form holes in another portion of bone, such as the second non-articular surface NS-2 below the glenoid rim GR.

The guide 243 may incorporate various metallic and/or non-metallic materials. Metallic materials may include a surgical grade steel or titanium. Non-metallic materials may include one or more polymers. The guide 243 may be formed utilizing various manufacturing techniques, including additive manufacturing (e.g., printing), injection molding, casting and machining.

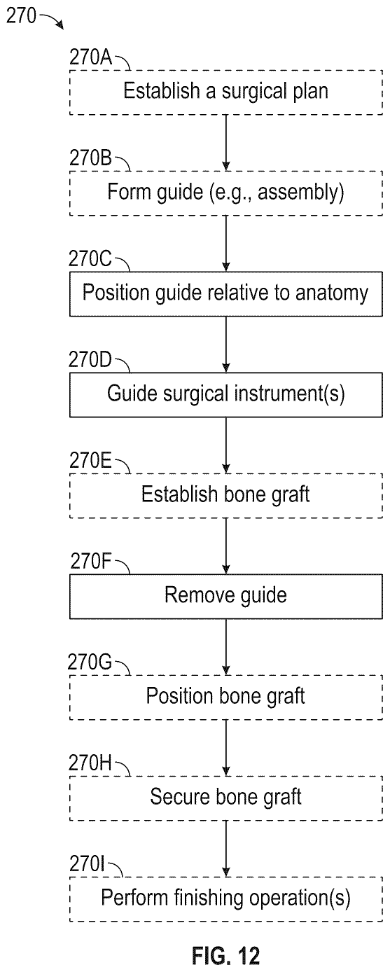

FIG. 12 discloses a method of planning and performing an orthopaedic procedure in a flowchart 270. The method 270 may be utilized to pre-operatively plan and perform an arthroplasty for restoring functionality to shoulders, ankles, knees, hips and other joints having bone loss or erosion along articular surfaces of the joint. The method 270 may be utilized to dimension one or more assemblies and other instruments, which may be based on a determined position and/or amount of bone loss. The method 270 may be utilized to generate one or more dimensions associated with instruments (e.g., assemblies) for performing an orthopaedic procedure according to an associated surgical plan for a patient. The dimensions may be utilized to fabricate or otherwise form an instrument, which may have one or more patient-specific surfaces and/or other dimensions associated with the surgical plan for a patient. The method 270 may be utilized with any of the instrumentation and guide models disclosed herein, including the guide 243 and/or guide model 141. Fewer or additional steps than are recited below could be performed within the scope of this disclosure, and the recited order of steps is not intended to limit this disclosure. Reference is made to the system 120, user interface 142 and guide 243.

Referring to FIGS. 2 and 7, with continuing reference to FIG. 12, a surgical plan 131 may be established at block 270A. The surgical plan 131 may be associated with a respective patient. Establishing the surgical plan 131 may include establishing at least one guide model 141 and/or an associated configuration. The surgical plan 131 and guide model 141 may be established utilizing any of the techniques disclosed herein.

At block 270B, a physical guide (e.g., assembly) may be fabricated or otherwise formed. The guide 243 may be formed according to a guide model 141. The guide 243 may include any of the features disclosed herein. The guide 243 may be formed utilizing any of the techniques disclosed herein.

Referring to FIGS. 13-14, with continuing reference to FIG. 12, the guide 243 may be positioned relative to an anatomy A of a patient at block 270C. The anatomy A may include any of the anatomy disclosed herein. The anatomy A may include a bone B, such as a scapula S associated with a shoulder joint. The guide 243 may include a first reference member 248 and/or a second reference member 260. Positioning the guide 243 may include positioning a first patient-specific contact surface 252 of the first reference member 248 on a first non-articular surface NS-1 of the bone B. The non-articular surface NS-1 may be associated with a coracoid process CP of the patient, or another portion of the scapula S, such as the acromion process AP. Positioning the guide 243 may include positioning a second patient-specific contact surface 264 of the second reference member 260 on an articular surface AS of the bone B (e.g., FIG. 13). The articular surface AS may be associated with a glenoid G of the patient.

The second contact surface 264 may be dimensioned to follow a second non-articular surface NS-2 of the bone B below a rim GR of the bone B bounding the articular surface AS, such as the glenoid rim GR. The second contact surface 264 may extend along a second recess 265 of the second reference member 260. Positioning the second contact surface 264 may occur such that the rim GR may be captured in the second recess 265 (e.g., FIG. 13).

The first patient-specific contact surface 252 may be dimensioned according to a first patient-specific contour CON-1 of the anatomy A. The second patient-specific contact surface 264 may be dimensioned according to a second patient-specific contour CON-2 of the anatomy A. The second patient-specific contour CON-2 may be associated with an anterior portion of the glenoid G. Positioning the first patient-specific contact surface 252 may occur such that the first patient-specific contact surface 252 may be positioned on a non-articular portion of the scapula S, such as the coracoid process CP, or the acromion process AP. Positioning the second patient-specific contact surface 264 may occur such that the second patient-specific contact surface 264 may be positioned on an articular and/or non-articular portion of the scapula S, such as an anterior portion of the glenoid G. The second contour CON-2 may be associated with different portions of the glenoid G, including posterior, superior and/or inferior portion(s) the glenoid G. The recesses 253, 265 may be dimensioned to capture portions of the anatomy A associated with the respective patient-specific contours CON-1, CON-2.

The reference members 248, 260 may be interconnected by a linking member 266. The linking member 266 may fix a relative position between the reference members 248, 260. The linking member 266 may be substantially rigid or may be at least partially flexible, which may facilitate positioning the reference members 248, 260 on the bone B. The reference members 248, 260 may be integrally formed with each other and/or the linking member 266. In other implementations, the reference members 248, 260 and linking members 266 may be separate and distinct components, which may be fixedly attached or otherwise secured to each other.

At block 270D, one or more surgical instruments SI may be guided relative to the anatomy A using the guide 243. The surgical instruments SI may include any of the surgical instruments disclosed herein. In implementations, the surgical instruments SI may include (e.g., physical) instruments SI-1 to SI-6, which may be associated with the (e.g., virtual) surgical instruments SI-1 to SI-6 utilized to establish the guide model 141.

Guide features 254 of the first and/or second reference members 248, 260 may be utilized to guide the respective instruments SI, including the passage(s) 256 and/or slot(s) 258 (e.g., FIG. 14). The guide features 254 may be positioned relative to the anatomy A to implement a respective surgical plan 131 associated with the patient (e.g., FIG. 2). Block 270D may include removing tissue from the anatomy A, including bone. In implementations, the instrument(s) SI-1, SI-2, SI-4 and/or SI-5 may be used to drill or otherwise form one or more holes in the bone B. The instrument(s) SI-1, SI-2, SI-4 and/or SI-5 may be different instruments or may be a single instrument. One or more cutting instruments (e.g., saw blades) such as the instrument SI-3 may be utilized to perform one or more cuts in the bone B. The cut may extend partially or completely through the bone B.

The first and/or second reference members 248, 260 may include one or more passages 256, such as passages 256-1, 256-2, 256-3, 256-4 (e.g., FIGS. 9-11). Block 270D may include guiding instrument(s) SI through the passage(s) 256 to form one or more holes in the first non-articular surface NS-1, the second non-articular surface NS-2, and/or the articular surface AS.

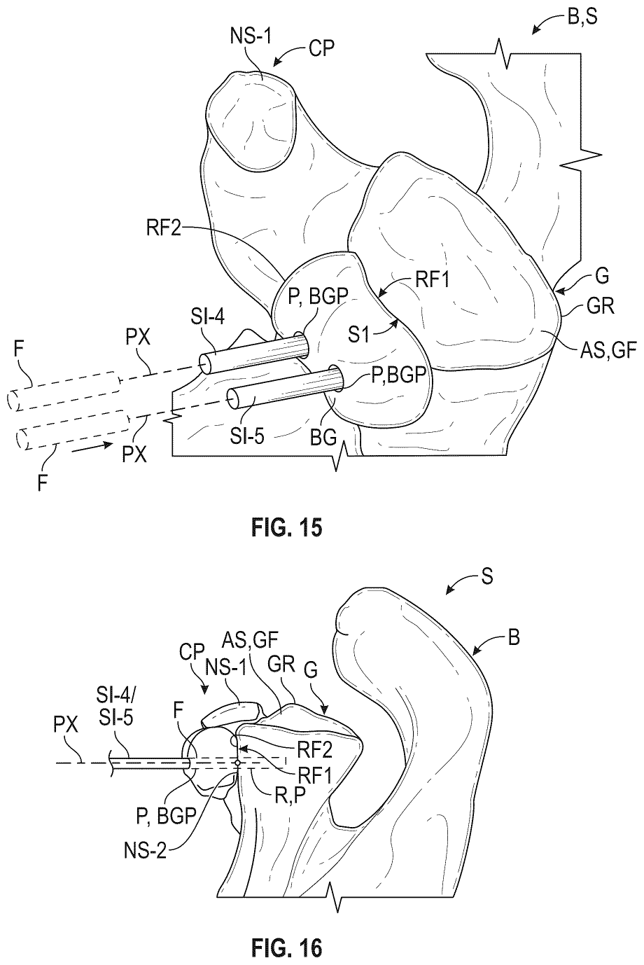

Referring to FIGS. 15-16, with continuing reference to FIGS. 12-14, block 270D may include forming one or more recesses R in the bone B (e.g., FIG. 16). The recesses R may be formed utilizing any of the techniques disclosed herein. The recesses R may have various geometries, including apertures, holes and passages, such as passages P. The passages P may be associated with one or more passages BPG in a bone graft BG.

The first reference member 248 may include a first set of passages 256-1, 256-2, and the second reference member 260 may include a second set of passages 256-3, 256-4 (e.g., FIGS. 9-11). Block 270D may include guiding the instrument(s) SI-1, SI-2 through the first set of passages 256-1, 256-2 to drill or otherwise form a first set of passages P in a portion of the bone B, which may be associated with the bone graft BG. The passages P may be associated with respective passages BGP in the bone graft BG. Forming the passages BGP prior to severing the portion of B may improve accuracy in forming the passages BGP, reduce the need for additional instrumentation, and decrease a duration of the surgical procedure. Block 270D may include guiding the instrument(s) SI-4, SI-5 through the second set of passages 256-3, 256-4 to drill or otherwise form a second set of passages P in another portion of the bone B, such as the glenoid G.

At block 270E, the bone graft BG may be established. In implementations, guiding the surgical instrument(s) SI at block 270D may occur to remove a portion BP of the bone B to establish the bone graft BG (e.g., FIGS. 13-14). The portion BP of bone B may be resected to establish the bone graft BG. The portion BP of bone B may be removed to establish the bone graft BG in response to guiding a cutting instrument (e.g., saw blade) SI-3 along a first guide (e.g., resection) surface 257 of the first reference member 248 (e.g., FIG. 14).

The bone graft BG may be shaped, dimensioned or otherwise prepared utilizing any of the techniques disclosed herein. The bone graft BG may be sized and/or shaped based on a determined bone loss along the respective bone B. In implementations, a portion of the bone graft BG may be removed to establish a first resection face RF1, which may occur in response to resecting the bone graft BG. Another portion of the bone B may be removed to establish a second resection face REF2 (FIG. 16), which may occur in response to guiding a (e.g., cutting) instrument SI along a resection surface of the second reference member 260. The removed portion of bone B adjacent to the second resection face REF2 may be associated with erosion along, or otherwise adjacent to, the glenoid rim GR. The resection faces RF1, RF2 may have complementary geometry such that the bone graft BG may be seated against the bone B. The surgeon may shape the bone graft BG and/or bone B with other surgical instrument(s) to establish the resection faces RF1, RF2, such as an osteotome or saw blade.

At block 270F, the guide 243 may be removed from the surgical site. The guide 243 may be removed after severing (e.g., harvesting) the portion BP of the bone B to establish the bone graft BG, forming the recesses R, passages P, BGP and/or resection faces RF1 and/or RF2, and/or otherwise modifying (e.g., preparing) the bone B.

At block 270G, the bone graft BG may be positioned relative to another portion of the bone B and/or anatomy A. The bone graft BG may be positioned in abutment with the bone B adjacent to the articular surface AS. The bone graft BG may be positioned utilizing any of the techniques disclosed herein. Placement of the bone graft BG may be specified in the surgical plan 131. Block 270G may include positioning the resection faces RF1, RF2 in abutment (e.g., FIG. 16). The passages BGP in the bone graft BG may be formed prior to, during or subsequent to positioning the bone graft BG relative to the bone B.

Positioning the bone graft BG may occur such that respective pairs of passages (e.g., holes) P in the bone graft BG and passages (e.g., holes) P in the bone B may be at least partially aligned with each other (e.g., FIG. 16). In implementations, the respective pairs of passages P in the bone graft BG and bone B may be substantially collinear with each other. Respective pairs of the passages P, BGP in the bone B and bone graft BGP may be substantially aligned with each other when the bone graft BG is positioned according to the surgical plan 131.

At block 270H, the bone graft BG may be secured to the bone B and/or another portion of the anatomy A. Various techniques may be utilized to secure the bone graft BG. In implementations, fastener(s) F may be positioned through the respective pair(s) of the passages (e.g., holes) P, BGP to secure the bone graft BG to the bone B (e.g., FIG. 16). The passages P may include pilot holes, which may extend through a cortex of the glenoid G.

One or more finishing operations may be performed at block 270H. The finishing operations may include closing an incision at the surgical site.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A guide for an orthopaedic procedure comprising:
a first reference member including a first body having a first contact surface dimensioned according to a first patient-specific contour associated with a first non-articular surface of an anatomy, and the first body including at least one guide feature dimensioned to guide a surgical instrument, the at least one guide feature including a resection surface that extends along a resection plane;
a second reference member spaced apart from the first reference member, the second reference member including a second body having a second contact surface dimensioned according to a second patient-specific contour associated with an articular surface of the anatomy; and
a linking member interconnecting the first and second reference members.

2. The guide as recited in claim 1, wherein:
the at least one guide feature includes a resection slot that extends along the resection surface.

3. The guide as recited in claim 1, wherein:
the linking member is dimensioned to span across a space between the articular surface and the first non-articular surface.

4. The guide as recited in claim 1, wherein:
the first contact surface extends along a first recess in the first body, and the first recess is dimensioned to receive a portion of bone associated with the first patient-specific contour.

5. The guide as recited in claim 4, wherein:
the resection plane intersects the first recess.

6. The guide as recited in claim 1, wherein:
the at least one guide feature of the first body includes a first set of passages dimensioned to receive a surgical instrument; and
the second body includes a second set of passages dimensioned to receive a surgical instrument.

7. The guide as recited in claim 6, wherein:
the second set of passages are established in the second body according to a placement of the first set of passages in the first body.

8. The guide as recited in claim 1, wherein:
the second contact surface extends along a second recess in the second body, and the second recess is dimensioned to receive a portion of bone associated with the second patient-specific contour;
the second patient-specific contour is associated with a second non-articular surface of the anatomy; and
the second recess is dimensioned to capture a rim bounding the articular surface of the anatomy such that the second contact surface abuts the second non-articular surface.

9. (canceled)

10. The guide as recited in claim 1, wherein:
the second body includes a passage, and the passage is dimensioned to guide an instrument to engage the anatomy below a rim bounding the articular surface of the anatomy.

11. The guide as recited in claim 1, wherein the first and second reference members are integrally formed with the linking member.

12. The guide as recited in claim 1, wherein the first patient-specific contour is associated with a coracoid process, and the second patient-specific contour is associated with the articular surface of a glenoid.

13. (canceled)

14. A guide for an orthopaedic procedure comprising:
a first reference member including a first body having a first contact surface dimensioned according to a first patient-specific contour associated with a first non-articular surface of an anatomy, wherein the first body includes a first passage having an opening along the first contact surface;
a second reference member spaced apart from the first reference member, the second reference member including a second body having a second contact surface dimensioned according to a second patient-specific contour associated with an articular surface of the anatomy, wherein the second body includes a second passage established in the second body according to a placement of the first passage in the first body; and
a linking member interconnecting the first and second reference members.

15. The guide as recited in claim 14, wherein the first and second passages are dimensioned to receive a drill instrument.

16. (canceled)

17. The guide as recited in claim 14, wherein at least one of the first body and the second body includes a resection surface dimensioned to guide a cutting instrument.

18. The guide as recited in claim 14, wherein the first and second reference members are integrally formed with the linking member.

19. The guide as recited in claim 14, wherein the first patient-specific contour is associated with a coracoid process, and the second patient-specific contour is associated with the articular surface of a glenoid.

20. (canceled)

21. A method of performing an orthopaedic procedure comprising:
positioning a first patient-specific contact surface of a first reference member on a first non-articular surface of a bone of a patient;
positioning a second patient-specific contact surface of a second reference member on an articular surface of the bone, wherein the first and second reference members are interconnected by a linking member;
removing a portion of the bone to establish a bone graft in response to guiding a cutting instrument along a first resection surface of the first reference member; and
positioning the bone graft on the bone adjacent to the articular surface.

22. The method as recited in claim 21, further comprising:
drilling a first set of holes in the portion of the bone through a first set of passages of the first reference member; and
drilling a second set of holes in the bone through a second set of passages of the second reference member;
wherein the step of positioning the bone graft occurs such that respective pairs of holes of the first and second sets of holes are at least partially aligned with each other.

23-24. (canceled)

25. The method as recited in claim 21, further comprising:
removing a portion of the bone graft to establish a first resection face;
removing another portion of the bone to establish a second resection face in response to guiding a cutting instrument along a second resection surface of the second reference member; and
positioning the first and second resection faces in abutment.

26. The method as recited in claim 21, wherein the articular surface is associated with a glenoid of the patient, and the first non-articular surface is associated with a coracoid process of the patient.

27-30. (canceled)
